(19) United States
(12) Patent Application Publication
(10) Pub. No.: US 2026/0306534 A1
(43) Pub. Date: Oct. 8, 2026

(54) A FLOATING DRUG DELIVERY SYSTEM FOR SOLID FORMS OF SALTS USED IN BOWEL CLEANSING PRODUCTS

(71) Applicant: Dark Canyon Laboratories, LLC, Rapid City, SD (US)

(72) Inventor: Dale R. Bachwich, Rapid City, SD (US)

(21) Appl. No.: 19/480,728

(22) PCT Filed: May 3, 2024
(86) PCT No.: PCT/US2024/027709

§ 371 Date: Oct. 31, 2025

Related U.S. Application Data
(60) Provisional application No. 63/499,828, filed on May 3, 2023.

(51) Int. Cl. A61K 33/14; A61K 9/48; A61K 33/04; A61P 1/10

(52) U.S. Cl. CPC A61K 33/14; A61K 9/4866; A61K 33/04; A61P 1/10

(57) ABSTRACT

The present disclosure provides systems, kits, and methods for preparation prior to a colonoscopy or other colon-related gastrointestinal procedure. In particular, the present invention provides a drug delivery system for bowel cleansing products and procedures. The drug delivery system comprises a capsule or tablet assembly that can be loaded with salts and other associated medications used in bowel cleansing products and yet float in the stomach after ingestion, due to a density of 1.004 gm/ml or less. This drug delivery system avoids large collections of salt in proximity to the gastric mucosa during the disintegration phase, thus reducing the risk of direct gastric mucosal injury that can occur with solid bowel cleansing products.

### CROSS-REFERENCE TO RELATED APPLICATIONS

This application claims the benefit of U.S. Provisional Application No. 63/499,828, filed May 3, 2023, the content of which is herein incorporated by reference in its entirety.

### FIELD OF THE INVENTION

The present disclosure provides systems, kits, and methods for preparation prior to a colonoscopy or other gastrointestinal procedure. In particular, the present disclosure provides a drug delivery system for bowel cleansing products and procedures.

### BACKGROUND

Bowel cleansing in the preparation for colonoscopy remains a significant barrier for patients and all other stakeholders in the use of colonoscopy for colorectal cancer prevention programs. Commercially available bowel cleansing products expressly for colonoscopy preparation were first approved for marketing by the FDA in 1984. The first product, GoLYTELY®, was safe, efficacious, but not well-tolerated by patients due to its significant volume (4 liters) and salty taste. The generic form of GoLYTELY® is referred to as polyethylene glycol and electrolyte solution, or PEG-ELS.

Several products have been developed in hopes of improving patient tolerability by altering the salt content of the product, adding flavorings, and reducing the volume of salt solution required. Additional efforts at improving tolerability have focused on delivering all or part of the medication for bowel cleansing in a tablet or capsule. While these products have improved patient tolerability, they remain imperfect for a variety of reasons.

The first tableted product for colonoscopy preparation was Diacol®, a 2.0 gm tablet containing 1.5 gm of sodium phosphate that was approved for marketing by the FDA in 2000 and marketed under the name Visicol®. Visicol® was better tolerated than PEG-ELS, but there was a problem with one of the excipients, microcrystalline cellulose, which left a residue in the colon that impeded the inspection phase of colonoscopy. Visicol® was reformulated to eliminate this excipient and marketed in the United States under the name Osmoprep®.

Post-marketing studies of Osmoprep® revealed that approximately 40% of patients using Osmoprep® reported nausea, 10% reported vomiting, and 10% developed erosive gastritis and/or gastric ulceration. Nevertheless, patients reported that this experience was an improvement over previous methods for colonoscopy preparation. More concerning was the growing realization that some patients developed renal failure after using Osmoprep® and other sodium phosphate products for colonoscopy preparation, due to the nephrotoxic effects of a large phosphate load contained in these products. Consequently, the FDA issued a black box warning for Osmoprep® because of its nephrotoxicity. GI Societies now discourage the use of sodium phosphate products for colonoscopy preparation.

Following the recognition of the nephrotoxicity of sodium phosphate, at least two encapsulated or tableted products have been developed for bowel cleansing prior to colonoscopy. Both SUTAB® and DCL-101 use solid forms of sodium sulfate as an osmotic agent for bowel cleansing as sodium sulfate is not nephrotoxic. SUTAB® is a tablet containing 1.479 g sodium sulfate, 0.225 g magnesium sulfate, and 0.188 g potassium chloride. It was approved by the FDA for marketing in 2020.

At the 2021 Annual Meeting of the American College of Gastroenterology (ACG), a poster reported a retrospective study of 33 patients who had received this newly approved tablet, generically known as oral sulfate tablet (OST). This group was compared to 94 patients who received a liquid bowel cleansing product over the same period. Twenty-seven (82%) of the thirty-three patients receiving OST had moderate to severe erosive gastritis characterized by adherent blood clots, mucosal edema/congestion, and florid erosions with or without ulcerations mostly over the greater curvature of the gastric body. This compared with 23% of patients who had erosive gastritis after taking a liquid preparation in this same study. A year later, at the 2022 ACG Annual Meeting, three new posters were presented highlighting the high rate (50-80%) of significant erosive gastritis and gastric ulceration in patients after the use of OST.

### SUMMARY

Disclosed herein are floating drug delivery systems (FDDS) comprising a floating capsule(s) and/or tablet(s) containing one or more active agents used in bowel cleansing. In some embodiments, the FDDS demonstrates rapid disintegration and/or full solubility of the one or more active agents.

In some embodiments, the floating capsule and/or tablet comprises floatation materials distributed along the long axis of the capsule and/or tablet. In some embodiments the floatation materials comprise one or more of a hollow interior capsule, hollow granules, and foam.

In some embodiments, the one or more active agents comprise sodium bicarbonate, sodium chloride, potassium chloride, sodium sulfate, citric acid, sodium citrate, ascorbic acid, sodium ascorbate and combinations thereof.

In some embodiments, the one or more active agents comprise salts of magnesium. In some embodiments, the salts of magnesium comprise magnesium sulfate, magnesium oxide, or magnesium citrate.

In some embodiments, the active agents comprise one or more adjunctive medications. In some embodiments, the one or more adjunctive medications comprise simethicone, bisacodyl, linaclotide, plecanatide, misoprostol, omeprazole, ranitidine, sodium picosulfate, or combinations thereof.

Also disclosed herein are methods of preparing for a colon-related procedure comprising administration of one or more doses of the FDDS disclosed herein over a period of time prior to the colon-related procedure. In some embodiments, the period of time is at least 4 hours. In some embodiments, the period of time is 12 to 24 hours.

In some embodiments, the methods further comprise administering one or more osmotic agents. In some embodiments, the one or more osmotic agents comprise PEG-3350, PEG-3000, PEG-4000, L-glucose, mannitol, sorbitol, xylitol, lactulose, glycerol, or any combination thereof. In some embodiments, the one or more osmotic agents are administered following dissolution in a liquid.

In some embodiments, the methods further comprise receiving a colon-related or gastrointestinal medical procedure. In some embodiments, the colon-related or gastrointestinal medical procedure comprises colonoscopy, capsule endoscopy, balloon enteroscopy, endoluminal gastroplication, endoscopic ultrasound (EUS), esophagogastroduodenoscopy (EGD), endoscopic retrograde cholangiopancreatography (ERCP), esophageal pH exam, flexible sigmoidoscopy (Flex Sig), hydrogen breath test, liver biopsy, percutaneous endoscopic gastrostomy (PEG), biofeedback for anorectal dysfunction, intraoperative radiation therapy (IORT), diagnostic GI radiology, CT colonography, CT enterography, MR colonography, MR enterography, laparoscopic cholecystectomy, colectomy, hysterectomy, hemorrhoidectomy, herniorrhaphy, or NOTES (natural orifice transluminal endoscopic surgery).

Further disclosed herein are kits comprising one or more doses of an FDDS as disclosed herein and one or more doses of osmotic agents.

Other aspects and embodiments of the disclosure will be apparent in light of the following detailed description and accompanying figures.

### BRIEF DESCRIPTION OF THE DRAWINGS

FIG. 1 shows an exemplary floating capsule in which two empty gelatin size “5” capsules serve as floatation devices when placed inside a larger capsule. The formulation can be encapsulated in larger capsule interior surround the smaller capsules.

FIGS. 2A and 2B are images of standard capsules without a floatation mechanism (FIG. 2A) and exemplary floating capsules (FIG. 2B) in an equal volume of water demonstrating the reduced density of the exemplary floating capsules.

FIGS. 3A-3D are images taken by esophagogastroduodenoscopy of a subject stomach following intake of 72 DCL-101 standard capsules over 12 hours. FIG. 3A is an image of the gastric antrum. FIGS. 3B and 3C are images of the gastric body. FIG. 3D is an image of the pre-pyloric region.

FIGS. 4A-4E are images taken by esophagogastroduodenoscopy of a subject stomach following intake of 72 capsules containing the modified DCL-101 formulation lacking potassium chloride (KCl) over 12 hours. FIGS. 4A-4D are images of the gastric body. FIG. 4E is an image of the pre-pyloric region.

FIGS. 5A-5E are images taken by esophagogastroduodenoscopy of a subject stomach following intake of 72 exemplary floating capsules containing the original DCL-101 formulation over 12 hours. FIGS. 5A-5D are images of the gastric body. FIG. 5E is an image of the pre-pyloric region.

### DETAILED DESCRIPTION

DCL-101 is an encapsulated bowel cleansing product that is administered as a dilute PEG-3350 solution and salt capsules containing sodium sulfate, sodium chloride, sodium bicarbonate, and potassium chloride. While comparable to PEG-ELS in composition, safety, and efficacy, it is far more tolerable as it is taste-free. It was recently found that that some patients developed erosive gastritis after using DCL-101. A prevailing hypothesis in the field of gastroenterology was that potassium chloride was the driver of mucosal injury, however, a prototype capsule that did not contain potassium chloride was tried and the gastric injury was no different from the full DCL-101 formulation, suggesting that other forces were causing gastritis.

Both oral sulfate tablets (OSTs) and DCL-101 capsules are rather dense, denser than water. This may cause such tablets/capsules to settle to the dependent portion of the stomach while the tablet/capsule disintegrated, and the salts dissolved. This process could create a local environment of high salt concentration, high osmotic force, and lead to mucosal disruption and injury. To address this issue a floating capsule system, as described herein, was developed to allow the capsule contents to be dispersed over a greater surface area of the stomach, or even dissolved, rather than settling in one area of the stomach and causing mucosal injury.

Such systems, collectively referred to as Floating Drug Delivery Systems (FDDS), have been described for many years and have been found to be ineffective for drugs that cause stomach mucosal irritation. Therefore, while the initial thought was that FDDS would be useful for drugs that irritate the gastric mucosa, over 40 years of subsequent experience and teaching indicate the opposite. Learned practitioners in the field of bowel cleansers, such as the developers of OSTs, have not included a FDDS in their bowel cleansing products recently brought to market.

### Definitions

The terms “comprise(s),” “include(s),” “having,” “has,” “can,” “contain(s),” and variants thereof, as used herein, are intended to be open-ended transitional phrases, terms, or words that do not preclude the possibility of additional acts or structures. However, two or more copies are also contemplated. The singular forms “a,” “and,” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

A “subject” or “patient” may be human or non-human and may include, for example, animal strains or species used as “model systems” for research purposes as described herein. Likewise, patient may include either adults or juveniles (e.g., children). Moreover, patient may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish, and the like. In one embodiment, the mammal is a human.

As used herein, “treat,” “treating,” and the like mean a slowing, stopping, or reversing of progression of a disease or disorder. The term also includes a reversing of the progression of such a disease or disorder to a point of eliminating or greatly reducing the disease.

Unless otherwise defined herein, scientific, and technical terms used in connection with the present disclosure shall have the meanings that are commonly understood by those of ordinary skill in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

### Floating Drug Delivery System (FDDS)

Disclosed herein are floating drug delivery system (FDDS) comprising a floating capsule(s) and/or tablet(s) containing one or more active agents used in bowel cleansing. Rather than extending the time for disintegration of the capsule and dissolution of the salts as is typical for FDDS, the current technology uses a gelatin capsule with a disintegration time of 4-9 minutes, the same as the original DCL-101 product. In some embodiments, the FDDS demonstrates rapid disintegration and/or full solubility of the one or more active agents.

Prior FDDS designs featured an elaborate aperture or membrane system to retain the drug in the capsule, but the present technology does not require any such system and the medication is simply contained by the outer capsule. Disintegration and drug release at the top surface of a volume of gastric fluid has meaningfully different consequences for the gastric mucosa compared to disintegration and drug release occurring as the capsule sits static on the mucosa at the dependent portion of the stomach. The short capsule/tablet disintegration time is useful for medications used for bowel cleansing, where significant numbers of capsules and fluid volumes are used (6-12 capsules per hour, up to a liter an hour of fluid intake).

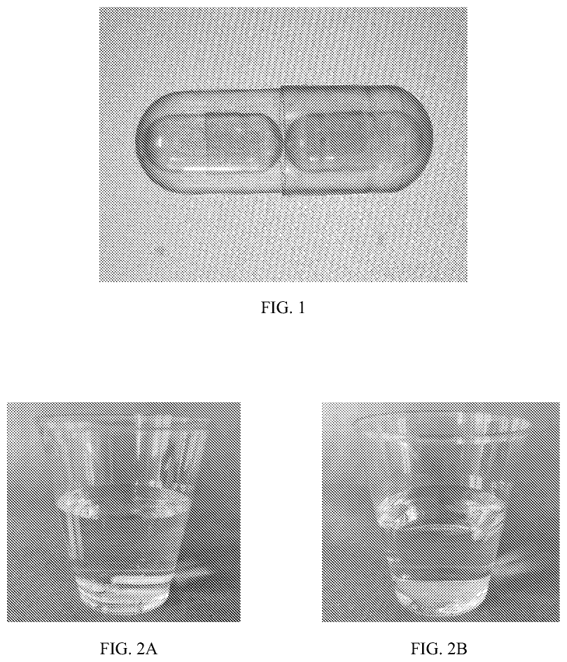

During in vitro studies it was found that it is desirable to have the floatation materials distributed along the long axis of the capsule, so that the capsule is oriented with the long axis parallel to the fluid surface during disintegration. This feature is distinct to other floatation mechanisms that intentionally weight one end of a tablet capsule so that the capsule floats with the long axis perpendicular to the surface. In some embodiments, two empty gelatin size “5” capsules serve as floatation devices (FIG. 1). In some embodiments, a gelatin “00” capsule, the main or outer capsule, is used to contain the salts and medications used in bowel cleansing and the floatation mechanism. Thus, the floatation mechanism minimizes development, regulatory, and production costs. Other dissolvable materials may be used as floatation devices including, but not limited to, hollow granules or foam inserted into both ends of the capsule. For example, tablets may include low-density foam or granules in the medication/excipient mix before molding or compression into tablets.

The capsules and/or tablets comprise one or more active agents used in bowel cleansing. In some embodiments, the one or more active agents comprise one or more electrolytes. In some embodiments, the one or more electrolytes comprises or consists of one or more salts. The one or more salts may comprise one or more cations (e.g., sodium, potassium, magnesium, calcium, etc.) and/or one or more anions (e.g., bicarbonate, chloride, phosphate, sulfate, etc.). In some embodiments, the one or more salts comprise or consist of: sodium sulfate, sodium bicarbonate, sodium chloride, sodium phosphate (e.g. monosodium phosphate, disodium phosphate, trisodium phosphate, etc.), potassium bicarbonate, potassium chloride, potassium phosphate, potassium sulfate, magnesium sulfate, magnesium bicarbonate, magnesium chloride, magnesium phosphates, calcium bicarbonate, calcium chloride, calcium phosphate, calcium sulfate, ascorbic acid, sodium ascorbate, or combinations thereof.

In some embodiments, the one or more salts comprise or consist of sodium bicarbonate, sodium chloride, potassium chloride, sodium sulfate, citric acid, sodium citrate, ascorbic acid and/or sodium ascorbate. In some embodiments, the one or more salts comprise or consist of salts of magnesium (e.g., magnesium sulfate, magnesium oxide, or magnesium citrate).

In some embodiments, sodium carbonate with or without other substances will be contained in capsules or tablets described herein in amounts of about 5 g or less to be ingested with each liter of co-administered solution. The sodium carbonate may be contained in the capsules or tablets at amounts of about 5 g, about 4.5 g, about 4 g, about 3.5 g, about 3 g, about 2.5 g, about 2 g, about 1.5 g, about 1 g, or less. In some embodiments, the amount of sodium carbonate is about 0.5 to about 5 g.

In some embodiments, sodium bicarbonate with or without other substances will be contained in capsules or tablets described herein in amounts of about 10 g or less to be ingested with each liter of co-administered solution. The sodium bicarbonate may be contained in the capsules or tablets at amounts of about 10 g, about 9.5 g, about 9 g, about 8.5 g, about 8 g, about 7.5 g, about 7 g, about 6.5 g, about 6 g, about 5.5 g, about 5 g, about 4.5 g, about 4 g, about 3.5 g, about 3 g, about 2.5 g, about 2 g, about 1.5 g, about 1 g, or less. In some embodiments, the amount of sodium carbonate is about 0.1 to about 10 g.

In some embodiments, sodium chloride with or without other substances will be contained in capsules or tablets described herein in amounts of about 6 g or less to be ingested with each liter of co-administered solution. The sodium chloride may be contained in the capsules or tablets in amounts of about 6 g, about 5.5 g, about 5 g, about 4.5 g, about 4 g, about 3.5 g, about 3 g, about 2.5 g, about 2 g, about 1.5 g, about 1 g, or less. In some embodiments, the amount of sodium chloride is about 0.1 to about 6 g.

In some embodiments, potassium chloride with or without other substances will be contained in capsules or tablets described herein in amounts of about 3 g or less to be ingested with each liter of co-administered solution. The potassium chloride may be contained in the capsules or tablets in amounts of about 3 g, about 2.5 g, about 2 g, about 1.5 g, about 1 g, or less. In some embodiments, the amount of potassium chloride is about 0.1 to about 3 g.

In some embodiments, sodium sulfate with or without other substances will be contained in capsules or tablets described herein in amounts of about 25 g or less to be ingested with each liter of co-administered. The sodium sulfate may be contained in the capsules or tablets at amounts of about 25 g, about 22 g, about 20 g, about 18 g, about 15 g, about 12 g, about 10 g, about 8 g, about 5 g, about 2 g, about 1 g, or less. In some embodiments, the amount of sodium sulfate is about 0.5 to about 25 g.

In some embodiments, magnesium sulfate with or without other substances will be contained in capsules or tablets described herein in amounts of about 25 g or less to be ingested with each liter of co-administered solution. The magnesium sulfate may be contained in the capsules or tablets at amounts of about 25 g, about 22 g, about 20 g, about 18 g, about 15 g, about 12 g, about 10 g, about 8 g, about 5 g, about 2 g, about 1 g, or less. In some embodiments, the amount of magnesium sulfate is about 0.5 to about 25 g.

In some embodiments, magnesium oxide with or without other substances will be contained in capsules or tablets described herein in amounts of about 25 g or less to be ingested with each liter of co-administered solution. The magnesium oxide may be contained in the capsules or tablets at amounts of about 25 g, about 22 g, about 20 g, about 18 g, about 15 g, about 12 g, about 10 g, about 8 g, about 5 g, about 2 g, about 1 g, or less. In some embodiments, the amount of magnesium oxide is about 0.5 to about 25 g.

In some embodiments, sodium ascorbate with or without other substances will be contained in capsules or tablets described herein in amounts of about 75 g or less to be ingested with each liter of co-administered solution. The sodium ascorbate may be contained in the capsules or tablets at amounts of about 75 g, about 70 g, about 65 g, about 60 g, about 55 g, about 50 g, about 45 g, about 40 g, about 35 g, about 30 g, about 25 g, about 20 g, about 15 g, about 10 g, about 5 g, about 1 g, or less. In some embodiments, the amount of sodium ascorbate is about 1 to about 75 g.

In some embodiments, ascorbic acid with or without other substances will be contained in capsules or tablets described herein in amounts of about 20 g or less to be ingested with each liter of co-administered solution. The ascorbic acid may be contained in the capsules or tablets at amounts of about 20 g, about 18 g, about 15 g, about 12 g, about 10 g, about 8 g, about 5 g, about 2 g, about 1 g, or less. In some embodiments, the amount of ascorbic acid is about 0.2 to about 20 g.

In some embodiments, citric acid with or without other substances will be contained in capsules or tablets described herein in amounts of about 20 g or less to be ingested with each liter of co-administered solution. The citric acid may be contained in the capsules or tablets at amounts of about 20 g, about 18 g, about 15 g, about 12 g, about 10 g, about 8 g, about 5 g, about 2 g, about 1 g, or less. In some embodiments, the amount of citric acid is about 0.2 to about 20 g.

In some embodiments, sodium citrate with or without other substances will be contained in capsules or tablets described herein in amounts of about 10 g or less to be ingested with each liter of co-administered solution. The sodium citrate may be contained in the capsules or tablets at amounts of about 10 g, about 9.5 g, about 9 g, about 8.5 g, about 8 g, about 7.5 g, about 7 g, about 6.5 g, about 6 g, about 5.5 g, about 5 g, about 4.5 g, about 4 g, about 3.5 g, about 3 g, about 2.5 g, about 2 g, about 1.5 g, about 1 g, or less. In some embodiments, the amount of sodium citrate is about 0.1 to about 10 g.

In some embodiments, magnesium citrate with or without other substances will be contained in capsules or tablets described herein in amounts of about 10 g or less to be ingested with each liter of co-administered solution. The magnesium citrate may be contained in the capsules or tablets at amounts of about 10 g, about 9.5 g, about 9 g, about 8.5 g, about 8 g, about 7.5 g, about 7 g, about 6.5 g, about 6 g, about 5.5 g, about 5 g, about 4.5 g, about 4 g, about 3.5 g, about 3 g, about 2.5 g, about 2 g, about 1.5 g, about 1 g, or less. In some embodiments, the amount of magnesium citrate is about 0.1 to about 10 g.

The amounts described above for ingestion with each liter of co-administered solution may be contained within a single capsule or tablet in the floating drug delivery system (FDDS) or may be spread out between multiple capsules or tablets to be administered at the same time. Each active agent may be contained within a single capsule or in combination with one or more additional active agents. For example, each active agent may be comprised within a single capsule having the amounts listed above for each active ingredient to be administered with each liter of solution. When administered in more than one liter, the FDDS may comprise one or more capsules and/or tablets in a single dose.

Salt formulations suitable for use with this technology include, but are not limited to, formulations previously described (see U.S. Pat. No. 10,898,515 and Bachwich D R, Lewis J D, Kowal V O, et al., Clin Transl Gastro 2020; 11:e00264). For example, DCL-101 (sodium sulfate 22.74 gm, sodium bicarbonate 6.74 gm, sodium chloride 5.86 gm, and potassium chloride 2.97 gm) or DCL-301 (sodium sulfate 15 gm, sodium chloride 5.4 gm, potassium chloride 2.03 gm, ascorbic acid 9.4 gm, and sodium ascorbate 11.8 gm).

In some embodiments, the one or more active agents further comprise one or more adjunctive medications. The adjunctive medications may be those useful for evacuating the colon, bowel cleansing, or for preventing complications associated with bowel cleansing, e.g., laxatives, agents for flatulence treatment/management, agents for management of stomach acids and ulcer formation. The adjunctive medications may hasten the elimination of digested and undigested remains of food in the large intestine and colon. In some embodiments, the adjunctive medications may be selected from: bulk-producing agents, stool softeners, surfactants, lubricants, emollients, hydrating agents, osmotics, saline solutions, hyperosmotic (e.g., PEG and/or sugar alcohols), stimulants, irritants, serotonin agonists, and the like. In some embodiments, the one or more adjunctive medications comprise simethicone, bisacodyl, linaclotide, plecanatide, misoprostil, omeprazole, ranitidine, sodium picosulfate, or combinations thereof.

In some embodiments, sodium picosulfate with or without other substances will be contained in capsules or tablets described herein in amounts of about 12 mg or less per liter of co-administered solution. The sodium picosulfate may be contained in the capsules or tablets at amounts of about 12 mg, about 11 mg, about 10 mg, about 9.5 mg, about 9 mg, about 8.5 mg, about 8 mg, about 7.5 mg, about 7 mg, about 6.5 mg, about 6 mg, about 5.5 mg, about 5 mg, about 4.5 mg, about 4 mg, about 3.5 mg, about 3 mg, about 2.5 mg, about 2 mg, about 1.5 mg, about 1 mg, or less. In some embodiments, the amount of sodium picosulfate is about 0.1 to about 12 mg.

In some embodiments, simethicone with or without other substances will be contained in capsules or tablets described herein in amounts of 700 mg or less per liter of co-administered solution. The simethicone may be contained in the capsules or tablets at amounts of about 700 mg, about 650 mg, about 600 mg, about 550 mg, about 500 mg, about 450 mg, about 400 mg, about 350 mg, about 300 mg, about 250 mg, about 200 mg, about 150 mg, about 100 mg, about 75 mg, about 50 mg, about 25 mg, about 10 mg, or less. In some embodiments, the amount of simethicone is about 1 to about 700 mg.

In some embodiments, bisacodyl with or without other substances will be contained in capsules or tablets described herein in amounts less than 12 mg per liter of co-administered solution. The bisacodyl may be contained in the capsules or tablets at amounts of about 12 mg, about 11 mg, about 10 mg, about 9.5 mg, about 9 mg, about 8.5 mg, about 8 mg, about 7.5 mg, about 7 mg, about 6.5 mg, about 6 mg, about 5.5 mg, about 5 mg, about 4.5 mg, about 4 mg, about 3.5 mg, about 3 mg, about 2.5 mg, about 2 mg, about 1.5 mg, about 1 mg, or less. In some embodiments, the amount of bisacodyl is about 0.1 to about 12 mg.

In some embodiments, linaclotide with or without other substances will be contained in capsules or tablets described herein in amounts less than 250 mcg per liter of co-administered solution. The linaclotide may be contained in the capsules or tablets at amounts of about 250 mcg, about 200 mcg, about 150 mcg, about 100 mcg, about 75 mcg, about 50 mcg, about 25 mcg, about 10 mcg, or less. In some embodiments, the amount of linaclotide is about 1 to about 250 mcg.

In some embodiments, plecanatide with or without other substances will be contained in capsules or tablets described herein in amounts less than 2 mg per liter of co-administered solution. The plecanatide may be contained in the capsules or tablets at amounts of about 2 mg, about 1.5 mg, about 1 mg, about 0.5 mg, about 0.25 mg, or less. In some embodiments, the amount of plecanatide is about 0.1 to about 2 mg.

In some embodiments, misoprostol with or without other substances will be contained in capsules or tablets described herein in amounts less than 500 mcg per liter of co-administered solution. The misoprostol may be contained in the capsules or tablets at amounts of about 500 mcg, about 450 mcg, about 400 mcg, about 350 mcg, about 300 mcg, about 250 mcg, about 200 mcg, about 150 mcg, about 100 mcg, about 75 mcg, about 50 mcg, about 25 mcg, about 10 mcg, or less. In some embodiments, the amount of misoprostol is about 1 to about 500 mcg.

In some embodiments, omeprazole with or without other substances will be contained in capsules or tablets described herein in amounts less than 15 mg per liter of co-administered solution. The omeprazole may be contained in the capsules or tablets at amounts of about 15 mg, about 14 mg, about 13 mg, about 12 mg, about 11 mg, about 10 mg, about 9.5 mg, about 9 mg, about 8.5 mg, about 8 mg, about 7.5 mg, about 7 mg, about 6.5 mg, about 6 mg, about 5.5 mg, about 5 mg, about 4.5 mg, about 4 mg, about 3.5 mg, about 3 mg, about 2.5 mg, about 2 mg, about 1.5 mg, about 1 mg, or less. In some embodiments, the amount of omeprazole is about 0.1 to about 12 mg.

In some embodiments, ranitidine with or without other substances will be contained in capsules or tablets described herein in amounts less than 15 mg per liter of co-administered solution. The ranitidine may be contained in the capsules or tablets at amounts of about 15 mg, about 14 mg, about 13 mg, about 12 mg, about 11 mg, about 10 mg, about 9.5 mg, about 9 mg, about 8.5 mg, about 8 mg, about 7.5 mg, about 7 mg, about 6.5 mg, about 6 mg, about 5.5 mg, about 5 mg, about 4.5 mg, about 4 mg, about 3.5 mg, about 3 mg, about 2.5 mg, about 2 mg, about 1.5 mg, about 1 mg, or less. In some embodiments, the amount of ranitidine is about 0.1 to about 12 mg.

### Kits

Also provided are kits comprising capsule(s), tablet(s), and/or the floating drug delivery system (FDDS) as described herein. For example, the kits may contain one or more doses of the sufficient for desired bowel cleansing procedure.

In some embodiments, the kits further comprise one or more doses of one or more osmotic agents. In some embodiments, the one or more osmotic agent comprises a polyethylene glycol (PEG) component. In some embodiments, the osmotic agent component comprises a poly(ethylene oxide) (PEO) or polyoxyethylene (POE) component. In some embodiments, the osmotic agent component comprises an L-glucose component. In some embodiments, the osmotic agent component comprises a sugar alcohol such as mannitol, sorbitol, or xylitol, lactulose, and/or glycerol. In some embodiments, the one or more osmotic agent comprises a mannitol component.

In some embodiments, the PEG component comprises PEG oligomers and/or polymers having a molecular weight or average molecular weight below 20,000 g/mol (e.g. <20000 g/mol, <15000 g/mol, <12000 g/mol, <10000 g/mol, <8000 g/mol, <6,000 g/mol, <4,000 g/mol, <2000 g/mol, <1000 g/mol, etc.). In some embodiments, the PEG oligomers and/or polymers have a molecular weight or average molecular weight above 105 g/mol (e.g., >200 g/mol, >300 g/mol, >400 g/mol, >500 g/mol, >750 g/mol, >1000 g/mol, >2000 g/mol, >4000 g/mol, >6000 g/mol, etc.). In some embodiments, the PEG oligomers and/or polymers have a molecular weight or average molecular weight greater than or equal to 1450 Daltons and less than or equal to 8000 Daltons. In some embodiments, the PEG oligomers and/or polymers of the present invention have a molecular weight or average molecular weight of approximately 3350 Daltons or of 3350 Daltons. In some embodiments, the PEG oligomers and/or polymers have low toxicity.

In some embodiments, the one or more osmotic agents do not contain salt or is substantially free of salt. In some embodiments, the one or more osmotic agents do not contain any salt other than salt that is provided as part of the osmotic agent, for example, as a byproduct of PEG manufacture. In some embodiments, the one or more osmotic agents are free from or substantially free from or functionally free (e.g., salts are present at a concentration insufficient to elicit a benefit to colon lavage procedures) from one or more of the salts: sodium sulfate, sodium bicarbonate, sodium chloride, sodium phosphate (e.g., monosodium phosphate, disodium phosphate, trisodium phosphate, etc.) potassium bicarbonate, potassium chloride, potassium phosphate, potassium sulfate, magnesium sulfate, magnesium bicarbonate, magnesium chloride, magnesium phosphates, calcium bicarbonate, calcium chloride, calcium phosphate, calcium sulfate, ascorbic acid, and sodium ascorbate.

In some embodiments, the one or more osmotic agents are provided in granular or powder form in doses ready to be dissolved in clear liquid (e.g., water or flavored drink). In some embodiments, doses of the one or more osmotic agents are pre-measured. In some embodiments, doses of the one or more osmotic agents are provided in doses to be dissolved in a specified volume of clear liquid (e.g., 250 ml, 500 ml, 1000 ml, 1500 ml, 2000 ml, etc.). In some embodiments, doses of one or more osmotic agents are provided in a single container with multiple compartments. In some embodiments, a specific number of doses of one or more osmotic agents are provided in a kit or package according to patient specific criteria (e.g., age, size, weight, sex, medical condition, species, etc.).

In some embodiments, the kits further comprise one or more doses of purgatives or purgative or adjunctive medications, as described above. The purgatives or purgative or adjunctive medications may be provided as separate doses, e.g., in individual bags, blister packs, cups, bottles, jars, envelopes, and/or containers, etc. In some embodiments, a specific number of doses of purgative are provided in a kit or package according to patient specific criteria (e.g., age, size, weight, sex, medical condition, species, etc.).

In some embodiments, kits are provided with flavored drink mix to use while consuming the described tablet(s) or capsule(s). In some embodiments, doses of flavored drink mix are pre-measured for a set quantity of liquid which is used to dissolve the one or more osmotic agents. In some embodiments, does of flavored drink mix are provided in individual bags, blister packs, cups, bottles, jars, envelopes, and/or containers, etc. In some embodiments, packages and/or kits of the present invention are provided with one or more different flavors of flavored drink mix (e.g. banana, cherry, strawberry, watermelon, root beer, passion fruit, berry, etc.). In some embodiments, flavored drink mix is not provided with kits and/or packages of the present invention. In some embodiments, an appropriate number of flavored drink mix doses are provided to correspond to the number of osmotic agent doses.

The kit may include instructions for any of the uses described herein, e.g., bowel cleansing and preparing for a colon-related procedure. In some embodiments instructions are provided with kits/packaging of the present invention. The instructions may include a CD-ROM or DVD (or other media source) providing a video set of detailed instructions in addition to printed instructions. This may also include a link to a web site providing additional information, answers to frequently asked questions, troubleshooting, and technical support.

The kits are provided in suitable packaging. Suitable packaging includes, but is not limited to, vials, bottles, jars, flexible packaging, and the like. The packaging may be single-use or multi-use packages. The packaging may be unit doses, bulk packages (e.g., multi-dose packages) or sub-unit doses. As described above, kits optionally may provide additional components such as interpretive information. Normally, the kit comprises a container and a label or package insert(s) on or associated with the container. In some embodiments, the disclosure provides articles of manufacture comprising contents of the kits described above.

The kit will typically be provided with its various components in one or more packages, e.g., a fiber-based, a cardboard, polymeric, or a Styrofoam box. The enclosure(s) can be configured so as to maintain a temperature differential between the interior and the exterior, for example, to provide insulating properties to keep the reagents at a preselected temperature for a preselected time. The packaging can be air-tight, waterproof (e.g., impermeable to changes in moisture or evaporation), and/or light-tight.

### Methods of Use

Disclosed herein are methods for subject preparation prior to a colon-related or gastrointestinal procedure. In some embodiments, the colon-related or gastrointestinal procedure includes, but is not limited to colonoscopy, capsule endoscopy, single- and double-balloon enteroscopy, endoluminal gastroplication, endoscopic ultrasound (EUS), esophagogastroduodenoscopy (EGD), endoscopic retrograde cholangiopancreatography (ERCP), esophageal pH exam, flexible sigmoidoscopy (Flex Sig), hydrogen breath test, liver biopsy, percutaneous endoscopic gastrostomy (PEG), biofeedback for anorectal dysfunction, intraoperative radiation therapy (IORT), diagnostic GI radiology, including barium enema, CT colonography, CT enterography, MR colonography, and MR enterography; surgical procedures including, but not limited to laparoscopic cholecystectomy (Gallbladder Removal), colectomy, hysterectomy, hemorrhoidectomy, herniorrhaphy, and NOTES (natural orifice transluminal endoscopic surgery).

In some embodiments, disclosed herein are methods for clearing digested and/or undigested food and/or stool from the colon and/or other portions of the gastrointestinal for health reasons, to restore health, to remove toxins (e.g., pollution, secondary smoking, harmful chemicals, pesticides, etc.), as a remedy for ailments (e.g., constipation, hepatic encephalopathy, acne, candida, brain fog, sluggishness, colonic dyssynergia, encopresis, constipative-predominant irritable bowel syndrome (IBS-C)), and to prevent disease.

In some embodiments, the methods comprise administration of one or more doses of a capsule(s), tablet(s), and/or the floating drug delivery system (FDDS) as described herein over a period of time prior to the colon-related or gastrointestinal procedure. In some embodiments, the period of time is at least four hours. In some embodiments, the period of time is at least twelve hours. For example, the period of time may be about 4 hours, about 6 hours, about 8 hours, about 10 hours, about 12 hours, about 14 hours, about 16 hours, about 18 hours, about 20 hours, about 22 hours, about 24 hours, or more. In some embodiments, the period of time is about 12 to about 24 hours.

In some embodiments, the methods further comprise administering of one or more osmotic agents. The osmotic agents described above for the disclosed kits are equally applicable to the disclosed methods. In some embodiments, the osmotic agents may be administered by dissolving the osmotic agents in the volume of liquid used to administer the disclosed capsule(s), tablet(s), and/or FDDS. The one or more osmotic agents may be provided in individually packaged doses or in bulk doses with a means for measuring into the individual doses.

In some embodiments, the methods further comprise administering one or more adjunctive medications, such as simethicone, bisacodyl, linaclotide, plecanatide, misoprostol, omeprazole, ranitidine, and sodium picosulfate.

Formulations and dosing regimens suitable for use in the disclosed methods include those of U.S. Pat. No. 10,898,515 and Bachwich D R, Lewis J D, Kowal V O, et al., Clin Transl Gastro 2020; 11:e00264), incorporated herein by reference in their entirety.

Exemplary formulations include, but are not limited to: DCL-101 (PEG-3350 236 gm dissolved in 4 L water, co-administered with: sodium sulfate 22.74 gm, sodium bicarbonate 6.74 gm, sodium chloride 5.86 gm, and potassium chloride 2.97 gm, in capsules described in this application) and DCL-301 (PEG-3350 200 gm dissolved in 3 L water, co-administered with: sodium sulfate 15 gm, sodium chloride 5.4 gm, potassium chloride 2.03 gm, ascorbic acid 9.4 gm, and sodium ascorbate 11.8 gm, in capsules described in this application).

An exemplary dosing regimen is as follows.

### One Day Prior to Procedure/Colonoscopy:

Step One: Ingest a low residue breakfast in the morning.
Step Two: Ingest a clear liquid lunch (juices, gelatin, ice pops, tea, coffee) mid-day. Avoid red and purple liquids, milk, and alcoholic beverages.
Step Three: Begin at 6 PM to 7 PM (early evening) to ingest the following:
   Mix 29.5 gm PEG-3350 with ~500 ml bottled water or high-quality tap water by shaking vigorously until the powder is dissolved. Drink the resulting solution over the course of 30 minutes using a straw or drinking cup.
   Consume capsule(s) (e.g., 6 capsules) as described herein during the 30 minutes that the PEG solution is consumed—one capsule every 80-90 mL.
   Repeat 4 times.

Water or clear, light-colored, non-caloric (containing no sugar or other calories) liquids may be consumed if the subject feels thirsty.

The day of the colonoscopy/procedure: (Beginning at 4 AM to 5 AM (early morning) if scheduled procedure time is before 12 PM noon or begin at 8 AM to 9 AM if scheduled procedure time is after 12 PM noon)
Mix 29.5 gm PEG-3350 with ~500 ml bottled water or high-quality tap water by shaking vigorously until the powder is dissolved. Drink the resulting solution over the course of 30 minutes using a straw or drinking cup.
Consume capsule(s) as described herein during the 30 minutes that the PEG solution is consumed—one capsule every 80-90 mL.
Repeat 4 times.

Water or clear, light-colored, non-caloric (containing no sugar or other calories) liquids may be consumed if the subject feels thirsty.

Finish consuming the kit at least two (2) hours prior to the scheduled colonoscopy/procedure time. No liquids should be consumed within two (2) hours of the scheduled colonoscopy/procedure time.

Subject should take any usual morning medications with a small sip of water once they have completed taking the prep kit.

### EXAMPLES

### Example 1

The buoyancy properties of DCL-101 using a standard capsule are compared to DCL-101 packaged in exemplary floating capsules as described herein. DCL-101 capsules contain:

DCL-101

| Components | Total Amount | | Per Capsule (n = 48) |
|---|---|---|---|
| Sodium sulfate | 22.74 | g | 0.474 g |
| Sodium bicarbonate | 6.74 | g | 0.140 g |
| Sodium chloride | 5.86 | g | 0.122 g |
| Potassium chloride | 2.97 | g | 0.062 g |

The DCL-101 formulation is currently packaged in “0” capsules. The average density of the filled capsules is approximately 1.2 gm/ml. This is confirmed by in vitro testing demonstrating that these capsules sink to the bottom of a container of water (FIG. 2A).

Placing these same capsule contents into “00” prototype capsules yield capsules with a density of 0.84 gm/ml, which float in a container of water (FIG. 2B). Note the long axis is parallel to the surface of the water.

### Example 2

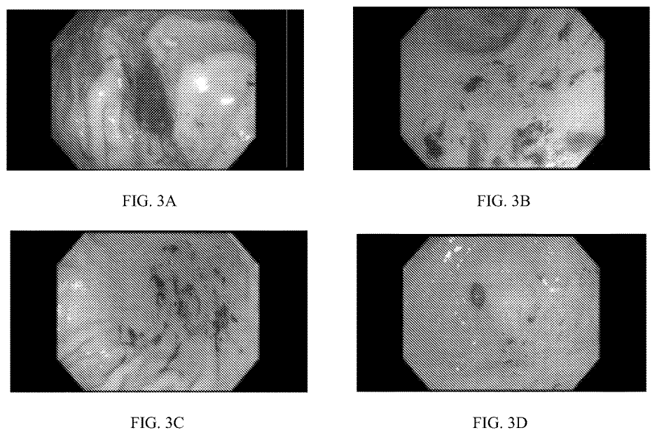

An informed subject consented to participate in a series of studies to test exemplary floating capsules as described herein. This subject underwent an esophagogastroduodenoscopy (EGD) after taking 72 DCL-101 standard capsules over 12 hours. Esophagogastroduodenoscopy revealed significant erosive gastritis (FIGS. 3A-3D). Note areas of bleeding or hemorrhage that appear as deep red to black spots on the stomach lining.

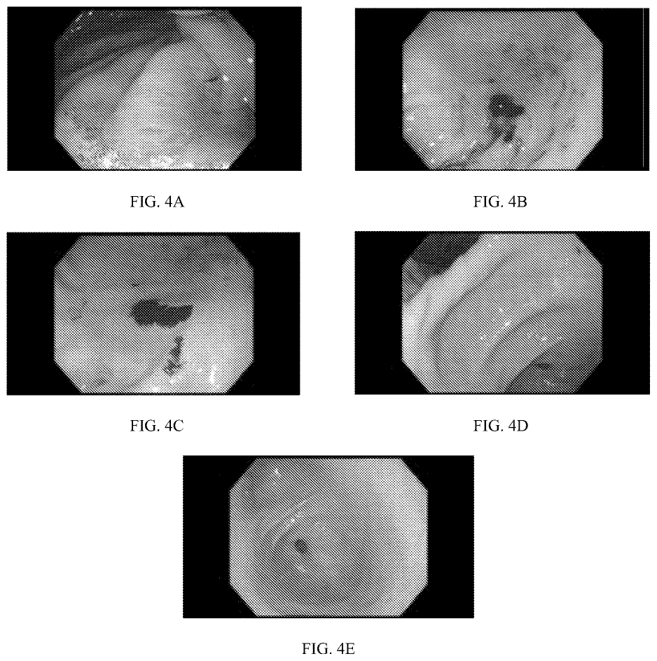

Twelve weeks later, the subject took 72 capsules containing the modified DCL-101 formulation lacking potassium chloride (KCl). Esophagogastroduodenoscopy revealed significant erosive gastritis (FIGS. 4A-4E) similar to that see with the standard capsules.

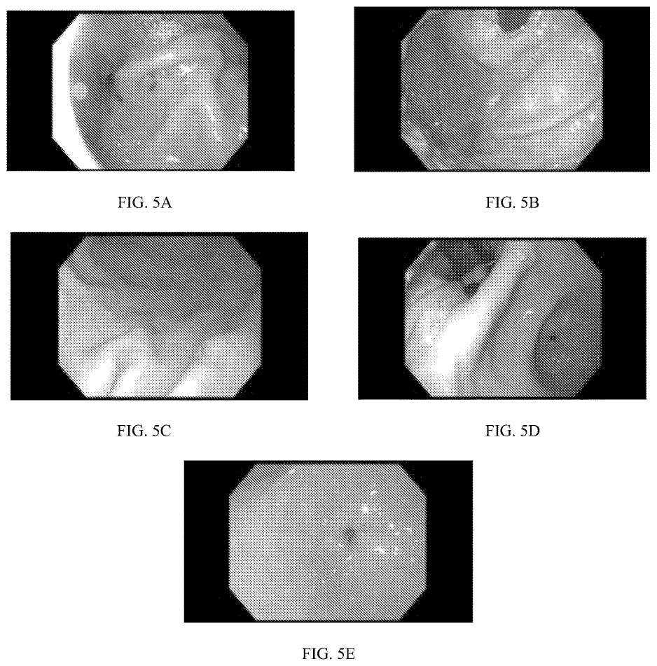

Four weeks later, the same subject took 72 exemplary floating capsules containing the original DCL-101 formulation in over 12 hours and underwent EGD revealing minimal erosive gastritis compared to earlier exams (FIGS. 5A-5E). Thus, the disclosed rapidly dissolving, floating drug delivery system is useful in ameliorating the gastric mucosal injury seen with solid forms of bowel cleansing products.

It is understood that the foregoing detailed descriptions are merely illustrative and are not to be taken as limitations upon the scope of the disclosure, which is defined solely by the appended claims and their equivalents.

All publications and patents mentioned in the above specification are herein incorporated by reference as if expressly set forth herein. Various changes and modifications to the disclosed embodiments will be apparent to those skilled in the art and may be made without departing from the spirit and scope thereof.

We claim:

1. A floating drug delivery system (FDDS) comprising a floating capsule(s) and/or tablet(s) containing one or more active agents used in bowel cleansing.

2. The FDDS of claim 1, wherein the FDDS demonstrates rapid disintegration and/or full solubility of the one or more active agents.

3. The FDDS of claim 1 or 2, wherein the floating capsule and/or tablet comprises floatation materials distributed along the long axis of the capsule and/or tablet.

4. The FDDS of claim 3, wherein the floatation materials comprise one or more of a hollow interior capsule, hollow granules, and foam.

5. The FDDS of any of claims 1-4, wherein the one or more active agents comprise sodium bicarbonate, sodium chloride, potassium chloride, sodium sulfate, citric acid, sodium citrate, ascorbic acid, sodium ascorbate and combinations thereof.

6. The FDDS of any of claims 1-5, wherein the one or more active agents comprise salts of magnesium.

7. The FDDS of claim 6, wherein the salts of magnesium comprise magnesium sulfate, magnesium oxide, or magnesium citrate.

8. The FDDS of any of claims 1-7, wherein the active agents comprise one or more adjunctive medications.

9. The FDDS of claim 8, wherein the one or more adjunctive medications comprise simethicone, bisacodyl, linaclotide, plecanatide, misoprostol, omeprazole, ranitidine, sodium picosulfate, or combinations thereof.

10. A method of preparing for a colon-related procedure comprising administration of one or more doses of an FDDS of any of claims 1-9 over a period of time prior to the colon-related procedure.

11. The method of claim 10, wherein the period of time is at least 4 hours.

12. The method of claim 10 or 11, wherein the period of time is 12 to 24 hours.

13. The method of any of claims 10-12, further comprising administering one or more osmotic agents.

14. The method of claim 13, wherein the one or more osmotic agents comprise PEG-3350, PEG-3000, PEG-4000, L-glucose, mannitol, sorbitol, xylitol, lactulose, glycerol, or any combination thereof.

15. The method of claim 13 or 14, wherein the one or more osmotic agents are administered following dissolution in a liquid.

16. The method of any of claims 10-15, further comprising receiving a colon-related or gastrointestinal medical procedure.

17. The method of claim 16, wherein the colon-related or gastrointestinal medical procedure comprises colonoscopy, capsule endoscopy, balloon enteroscopy, endoluminal gastroplication, endoscopic ultrasound (EUS), esophagogastroduodenoscopy (EGD), endoscopic retrograde cholangiopancreatography (ERCP), esophageal pH exam, flexible sigmoidoscopy (Flex Sig), hydrogen breath test, liver biopsy, percutaneous endoscopic gastrostomy (PEG), biofeedback for anorectal dysfunction, intraoperative radiation therapy (IORT), diagnostic GI radiology, CT colonography, CT enterography, MR colonography, MR enterography, laparoscopic cholecystectomy, colectomy, hysterectomy, hemorrhoidectomy, herniorrhaphy, or NOTES (natural orifice transluminal endoscopic surgery).

18. A kit comprising one or more doses of an FDDS of any of claims 1-9 and one or more doses of osmotic agents.
